(19) United States
(12) Patent Application Publication
(10) Pub. No.: US 2026/0069812 A1
(43) Pub. Date: Mar. 12, 2026

(54) NASAL CANNULA APPARATUS FOR OXYGEN DELIVERY AND RELATED METHODS

(71) Applicant: BRYAN MICHAEL LIN, ORLANDO, FL (US)

(72) Inventors: EDWARD DANIEL LIN, OSPREY, FL (US); SUZANNA LUI LIN, OSPREY, FL (US); BRYAN MICHAEL LIN, ORLANDO, FL (US); LIANG JIUN LIN, NEW TAIPEI CITY (TW); YUN CHIN HUANG, NEW TAIPEI CITY (TW)

(21) Appl. No.: 18/829,787

(22) Filed: Sep. 10, 2024

(51) Int. Cl. A61M 16/06; A61M 16/20

(52) U.S. Cl. CPC A61M 16/0672; A61M 16/209; A61M 2202/0007; A61M 2202/0208; A61M 2205/3303; A61M 2205/3327; A61M 2205/3344; A61M 2205/3368; A61M 2205/75; A61M 2210/0618; A61M 2230/43

(57) ABSTRACT

The nasal cannula apparatus includes a pair of nasal cannula connected to a body defining an internal body chamber. The body is adapted to be secured about the head of a user in engagement with the user generally between the nares and the upper lip and with the pair of nasal cannula engaged with the nares to fluidly communicate between the user's lungs and the body chamber. Respiratory gas is communicated into the body chamber for delivery to the user via the pair of nasal cannula. Outflow gas is communicated from the lungs via the pair of nasal cannula into the body chamber for discharge from the body chamber via a discharge tube. An inflow valve disposed within the body controls communication of respiratory gas into the body chamber. An outflow valve may be disposed within the body to control fluid communication between the body chamber and the discharge tube.

### BACKGROUND OF THE INVENTION

### Field of the Invention

This disclosure relates to apparatus and related methods for delivering oxygen in support of respiration in users, and more specifically, to nasal cannula operable to deliver oxygen support.

### Related Art

Shortness of breath from varying degrees of hypoxia (lack of oxygen) is one of the most common and potentially critical ailments confronting humans. Hypoxia may have a variety of underlying causes may be either acute or chronic. For example, acute hypoxia may be caused by bacterial or viral pneumonia, asthmatic exacerbation, pulmonary embolus, stroke, and severe trauma. Chronic hypoxia may be secondary to many cardiopulmonary pathologies including, for example, congestive heart failure, heart valve diseases, cardiomyopathy, congenital heart disease, COPD (chronic obstructive pulmonary diseases such as emphysema), pulmonary fibrosis, and other structural heart diseases.

Oxygen when inhaled first passes through the nasal pharynx then down the trachea to the bifurcating bronchi and bronchioles to the terminal bronchioles before reaching the alveolar spaces where oxygen exchange with pulmonary capillaries actually occurs. All of the gas conducting spaces beginning at the nose or mouth and ending just before the alveoli are referred to collectively as the “anatomical dead space” as no oxygen exchange takes place in this region. For an average adult, the anatomical dead space of approximately 150 ml accounts for about ⅓ of a typical normal breath (referred to as tidal volume, or TV) of about 450 ml. Therefore, only 300 ml of the inhaled 450 ml actually participates in alveolar oxygen exchange. This fact is of great importance in optimizing treatment for any hypoxic patient. During inspiration, the rate of air inflow begins slowly and quickly peaks (known as peak inspiratory flow rate, or PIFR) for about 1.5 seconds before slowing down and halting as the end of tidal volume is reached. During PIFR, the air intake rate may be 60 LPM or higher (around 1000 ml/second), but lasts only briefly. Therefore, a flow rate of up to about 1000 ml/second may be needed briefly during peak inspiratory flow in order to deliver a comfortable breath to the user.

Nasal cannula devices are the most commonly used device to provide supplemental oxygen. Typical nasal cannula devices comprise a soft PVC tubing worn around the ears and in fluid communication with dual nasal cannula that partially enter the nares to deliver oxygen flow into the nasopharynx. The tubing is in communication with an oxygen source to receive oxygen therefrom for delivery into the nasopharynx. The oxygen flow rate through the nasal cannula is typically around 2 LPM to 5 LPM, although flow up to 15 LPM may be given as hypoxia becomes more prominent. However, if the nasal cannula is supplying oxygen at 2, 5, 10 or 15 LPM, the deliverable oxygen is a mere 33 ml, 83 ml, 167 ml and 250 ml/second, respectively. So there may be a brief mismatch between peak demand (1000 ml/second PIFR) and supply when using the nasal cannula resulting in ambient air being inhaled in lieu of oxygen.

Nasal cannula devices may have several other drawbacks: (1) the jet flow of oxygen into the nasal pharynx via the loose fitting nasal prongs creates a Venturi effect that entrains ambient air that dilutes the oxygen concentration; (2) the oxygen flow to the nares is constant so that oxygen flows during expiration which wastes this oxygen; (3) the oxygen flow during expiration dilutes the expired gas rendering it difficult to obtain even a tracing, let alone an accurate measurement, of attributes of the expired gas such as EtCO₂ (end tidal CO₂) that is recognized as a critical parameter in determining the adequacy of ventilation. It may be difficult to measure other attributes of the expired gas because of the dilution of expired gas by the oxygen being supplied.

In the years since the nasal cannula was invented by Wilfred Jones in 1949, the above deficiencies in the nasal cannula remain under-recognized and uncorrected. Accordingly, there is a need for improved apparatus as well as related methods for oxygen delivery using nasal cannula.

### BRIEF SUMMARY OF THE INVENTION

These and other needs and disadvantages may be overcome by the apparatus and related methods disclosed herein. Additional improvements and advantages may be recognized by those of ordinary skill in the art upon study of the present disclosure.

In various aspects, the nasal cannula apparatus disclosed herein includes a body that defines a body chamber with the body being adapted to engage a user between nares and upper lip. The body includes an inflow port in fluid communication with the body chamber to communicate a respiratory gas into the body chamber, and an inflow valve is disposed within the body chamber in cooperation with the inflow port to control flow through the inflow port, in various aspects. The body includes an outflow port in fluid communication with the body chamber to communicate an outflow gas from the body chamber, in various aspects. A pair of nasal cannula is disposed about the body, and each nasal cannula of the pair of nasal cannula is adapted to engage occlusively a naris in order to communicate fluidly between the naris and the body chamber via the pair of nasal cannula, in various aspects. The inflow valve allows respiratory gas communication through the inflow port into the body chamber during inhalation and blocks respiratory gas communication through the inflow port into the body chamber during exhalation, in various aspects.

In certain aspects, an outflow valve is disposed within the body chamber in cooperation with the outflow port, and the outflow valve is configured to control fluid communication through the outflow port. For example, the outflow valve blocks fluid communication through the outflow port during inhalation and allows fluid communication from the body chamber through the outflow port during exhalation, in certain aspects.

Methods disclosed herein may include the step of attaching the body to the user. The body may be placed generally between the upper lip and the external nares of the user with the pair of nasal cannula operably engaged with the nares of the user, in various aspects. The pair of nasal cannula may be either occlusively or nonocclusively engaged with the nares, in various aspects.

Methods disclosed herein may include the step of positioning the inflow valve in an OPEN position by decreasing the chamber pressure pr within the body chamber to less than a supply pressure pₛ within the supply tube by the user inhaling respiratory gas from the body chamber through the pair of nasal cannulae. With the inflow valve in the OPEN position, respiratory gas flows from the supply tube into the body chamber and thence via the pair of nasal cannula into the nares for delivery to the lungs.

Methods disclosed herein may include the step of positioning the outflow valve in an CLOSED position by decreasing the chamber pressure P_{R} within the body chamber to less than a discharge pressure p_{d} within the discharge tube by the user inhaling respiratory gas from the body chamber through the nasal cannulae; the outflow valve in the CLOSED position blocking fluid communication between the body chamber and the discharge tube during inhalation.

Methods disclosed herein may include the step of positioning the inflow valve in the CLOSED position by increasing the chamber pressure P_{R} within the body chamber to greater than supply pressure pₛ within the supply tube by the user exhaling outflow gas from the lungs through the pair of nasal cannula into the body chamber; the inflow valve in the CLOSED position blocking the flow respiratory gas from the supply tube into the body chamber.

Methods disclosed herein may include the step of positioning the outflow valve in the OPEN position by increasing the chamber pressure P_{R} within the body chamber to greater than discharge pressure pa within the discharge tube by the user exhaling outflow gas from the lungs through the pair of nasal cannula into the body chamber; the outflow valve in the OPEN position allowing the flow of outflow gas from the body chamber into the discharge tube.

Methods disclosed herein may include the step of detecting an attribute of the outflow gas using a sensor in fluid cooperation with the outflow gas. The sensor may communicate data indicative of the attribute to a computer for processing and display, and may do so generally in real time either intermittently or continuously, in various aspects.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1 illustrates by perspective view an exemplary implementation of a nasal cannula apparatus in accordance with the present inventions;

FIG. 2A illustrates by cut-away elevation view portions of the exemplary implementation of the nasal cannula apparatus of FIG. 1;

FIG. 2B illustrates by plan view portions of the exemplary implementation of the nasal cannula apparatus of FIG. 1;

FIG. 2C illustrates by plan view portions of the exemplary implementation of the nasal cannula apparatus of FIG. 1;

FIG. 3A illustrates by perspective view portions of the exemplary implementation of the nasal cannula apparatus of FIG. 1;

FIG. 3B illustrates by cut-away view the portions of the exemplary implementation of the nasal cannula apparatus of FIG. 3A;

FIG. 4A illustrates by cut-away view an exemplary inflow valve of the exemplary implementation of the nasal cannula apparatus of FIG. 1 in an CLOSED position;

FIG. 4B illustrates by cut-away view the exemplary inflow valve of FIG. 4A in an OPEN position;

FIG. 5A illustrates by schematic diagram an operational state of the exemplary implementation of the nasal cannula apparatus of FIG. 1;

FIG. 5B illustrates by schematic diagram another operational state of the exemplary implementation of the nasal cannula apparatus of FIG. 1;

FIG. 5C illustrates by schematic diagram a third operational state of the exemplary implementation of the nasal cannula apparatus of FIG. 1;

FIG. 6A illustrates by schematic diagram an operational state of another exemplary implementation of a nasal cannula apparatus;

FIG. 6B illustrates by schematic diagram another operational state of the exemplary implementation of the nasal cannula apparatus of FIG. 6A; and,

FIG. 7 illustrates by process flow chart certain exemplary methods of operation of the exemplary respiratory therapy apparatus of FIGS. 1, 6A.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. Use herein of relative terms such as generally, about, approximately, essentially, may be indicative of engineering, manufacturing, or scientific tolerances such as ±0.1%, ±1%, +2.5%, ±5%, or other such tolerances, as would be recognized by those of ordinary skill in the art upon study of this disclosure.

### DETAILED DESCRIPTION OF THE INVENTION

The nasal cannula apparatus disclosed herein includes a body adapted to be secured about the head of a user in engagement with the user generally between the nares and the upper lip, in various aspects. At least portions of the body may in biased contact with the user's skin generally between the nares and the upper lip. In various aspects, the body defines an internal body chamber disposed with the body. The body, in various aspects, includes an inflow port in fluid communication with the body chamber to communicate a respiratory gas from a gas source into the body chamber and an outflow port in fluid communication with the body chamber to communicate an outflow gas from the body chamber for discharge. An inflow valve may cooperate with the inflow port to control the communication of respiratory gas through the inflow port into the body chamber. The inflow valve may be disposed within the body, in some aspects, or the inflow valve may be disposed in the fluid pathway between the gas source and the body chamber, in other aspects. A pair of nasal cannula in mechanical and fluid cooperation with the body is adapted to engage occlusively the nares in order to communicate fluidly between the naris and the body chamber via the nasal cannulae. The pair of nasal cannula communicate the respiratory gas from the body chamber to the lungs of the user via the nares including the nasal passages, thence, into the lungs, and the pair of nasal cannula communicate outflow gas from the lungs to the body chamber for discharge through the outflow port, in various aspects. In various aspects, because of the occlusive engagement of the pair of nasal cannula with the nares, there is generally neither dilution of the respiratory gas being delivered to the lungs by entrainment of ambient air in the respiratory gas nor dilution of the outflow gas by entrainment of ambient air in the outflow gas that, inter alia, may cause errors in detection of attributes of the outflow gas. Because the body is sized to be positioned generally between the nares and the upper lip, a volume of the body chamber is small so that mixing of respiratory gas and outflow gas within the body chamber is minimized resulting in the user substantially inhaling respiratory gas, not a mixture of respiratory gas with outflow gas, in various aspects.

In various aspects, the inflow valve allows respiratory gas communication through the inflow port into the body chamber during inhalation and blocks respiratory gas communication through the inflow port into the body chamber during exhalation. Certain aspects may include an outflow valve in cooperation with the outflow port to block fluid communication through the outflow port during inhalation and allow fluid communication from the body chamber through the outflow port during exhalation. The outflow valve may be disposed within the body, in some aspects, or the outflow valve may be positioned downstream of the body, in other aspects. The nasal cannula apparatus may include a sensor in fluid cooperation with the outflow port to detect an attribute of the outflow gas communicated from the body chamber through the outflow port, in various aspects. The nasal cannula apparatus disclosed herein may be used for oxygen supplementation of spontaneously breathing users, in various aspects. The nasal cannula apparatus may be configured to release moisture from the body chamber that may accumulate within the body chamber, in various aspects. Because the nasal cannula apparatus may be single use, in various aspects, disposal following use may aid infection control.

As used herein, a user is defined as a person to whom the nasal cannula apparatus is attached or to whom the nasal cannula apparatus is adapted for attachment. In certain aspects, a healthcare provider may employ the nasal cannula apparatus in treating the user. The healthcare provider may be, for example, a physician, physician's assistant, nurse, respiratory therapist, or other caregiver.

As used herein, the terms distal and proximal are defined from the point of view of the healthcare provider treating the user with the nasal cannula apparatus. A distal portion of the nasal cannula apparatus is oriented toward the user (e.g., the person being treated) while a proximal portion of the nasal cannula apparatus is oriented toward the healthcare provider (e.g., the person performing the treatment). In general, a distal portion of the nasal cannula apparatus is closest to the user (e.g., the patient) while a proximal portion of the nasal cannula apparatus is closest to the healthcare provider treating the user.

As used herein, ambient pressure p_{amb} refers to the pressure in a region surrounding the nasal cannula apparatus. For example, ambient pressure p_{amb} may refer to atmospheric pressure at the location of the nasal cannula apparatus, hull pressure within an aircraft where the nasal cannula apparatus is being utilized, or pressure maintained within a building or other structure where the nasal cannula apparatus is being utilized. Ambient pressure p_{amb} may vary, for example, with elevation or weather conditions. Unless specifically stated, pressure as used herein refers to gauge pressure, that is, pressure relative to ambient pressure p_{amb}. Pressures may be given as gauge pressures, so that positive pressures indicate pressures greater than ambient pressure p_{amb} and negative pressures indicate pressures less than ambient pressure p_{amb}. Ambient air refers to the air at the location where the nasal cannula apparatus is being utilized. Respiratory gas, as used herein, includes oxygen at a concentration greater than that of atmospheric air, which is approximately 21%. Respiratory gas may include various gas mixtures such as the oxygen and nitrogen mixture Nitrox, a mixture of 79% helium and 21% oxygen, such as Heliox, or various other non-atmospheric gas mixtures. Note that respiratory gas, oxygen, and similar terms may be used interchangeably herein. Inhalation (inspiration) is the act of drawing in gas into the lungs. Exhalation (expiration) is the act of expelling gas from the lungs to the exterior of the body. The terms inhalation and inspiration, as well as exhalation and expiration and similar terms may be used interchangeably herein. Naris and the plural form nares refer to the opening into the nasal passages.

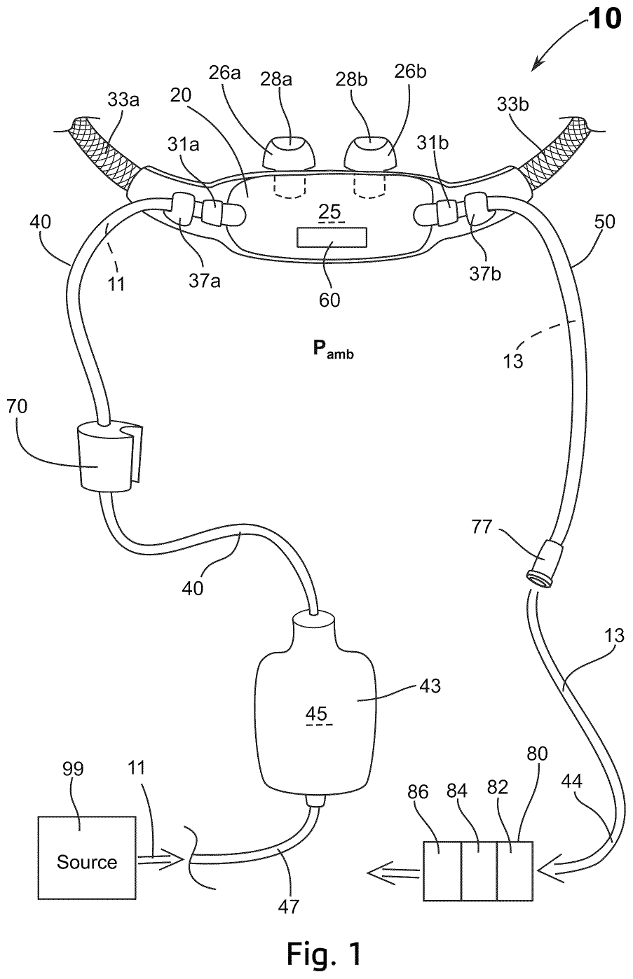

FIG. 1 illustrates exemplary nasal cannula apparatus 10 including body 20 that defines body chamber 25 within and nasal cannula 26a, 26b adapted to engage each naris including insertion into each nasal passage, respectively to fluidly communicate between the nasal passages and body chamber 25. Nasal cannula 26a, 26b may each engage the corresponding wall of the naris, and, thus, may occlude flow through the naris other than via the nasal cannula of nasal cannula 26a, 26b engaged with the naris thereby preventing dilution by atmospheric air of respiratory gas 11 being delivered through the nares and dilution of outflow gas 13 being exhaled through the nares.

Supply tube 40 communicates respiratory gas 11 into body chamber 25 for communication into the nares via nasal cannula 26a, 26b thence into the lungs as the user inhales. Discharge tube 50 communicates outflow gas 13 from body chamber 25, outflow gas 13 having been communicated, at least in part, from the lungs through the nares thence through nasal cannula 26a, 26b into body chamber 25 as the user exhales. Accordingly, outflow gas 13 may comprise exhalant from the user as well as other gaseous bodily emanations. Body 20 includes strap connectors 31a, 31b that engage straps 33a, 33b, respectively, that are configured to engage the head in order to secure body 20 to a user with nasal cannula 26a, 26b received within the nares and body 20 disposed generally between the upper lip (e.g., labium superius oris) and the external nares of the user. Straps 33a, 33b may be formed of an elastic material such as rubber. Body 20 may generally topically contact the user between the upper lip and nasal septum, for example, about the philtrum. In this implementation, clips 37a, 37b are disposed about body 20 to engage supply tube 40 and discharge tube 50, respectively, to support supply tube 40 and discharge tube 50 and to hold supply tube 40 and discharge tube 50 in attachment to body 20.

Supply pathway 47 fluidly communicates between source 99 and bag 43 to communicate respiratory gas 11 from source 99 into bag reservoir 45 of bag 43, as illustrated in FIG. 1.

Exemplary supply pathway 47 may comprise various piping, hoses, tubing, and other fluid pathways, as would be readily understood by those of ordinary skill in the art upon study of this disclosure. Gas source 99 may be, for example, a cylinder of compressed gas or mains oxygen.

In various implementations, gas source 99 may be an oxygen concentrator. The oxygen concentrator may, for example, supply 85-94% oxygen as respiratory gas 11 at a continuous flow of 5-10 L/min. In various implementations, gas source 99 may supply 85-94% oxygen as respiratory gas 11 at a continuous flow between about 2 L/min and about 15 L/min. In various implementations, gas source 99 may supply 85-94% oxygen as respiratory gas 11 at a continuous flow of around 10 L/min. The gas source 99 may be located in other than a hospital setting (e.g., in a home or residential setting), in certain implementations. Gas source 99 may include a pressure manifold that allows regulating of pressure within supply pathway 47 and may otherwise be configured as would be readily understood by those of ordinary skill in the art upon study of this disclosure.

As illustrated in FIG. 1, supply tube 40 communicates fluidly respiratory gas 11 from bag reservoir 45 into body chamber 25 of body 20 for delivery to the user. Optional bag 43 may be omitted in certain implementations, so that supply tube 40 communicates respiratory gas 11 from gas source 99 to body chamber 25 of body 20.

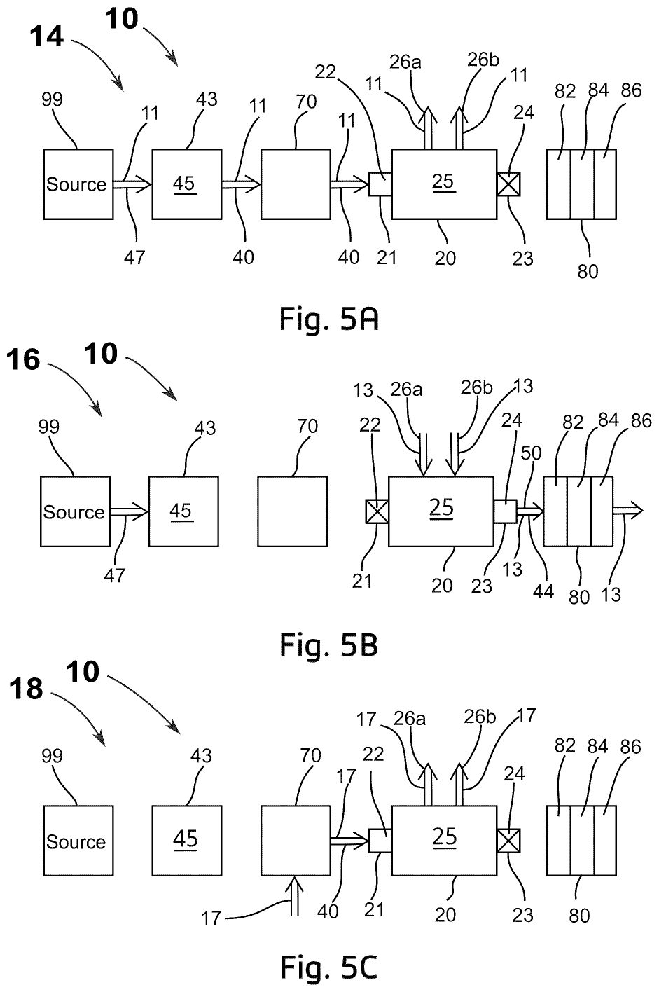

As illustrated in FIG. 1, anti-asphyxiation valve 70 cooperates with supply tube 40 to allow inflow of ambient air 17 (also see FIG. 5C) into supply tube 40 in the event of an insufficiency in supply of respiratory gas 11 from gas source 99 to body chamber 25. For example, low pressure (e.g., pressure below ambient pressure p_{amb}) in supply tube 40 that may be caused by the user attempting to breath while the supply of respiratory gas 11 is insufficient may initiate operation of anti-asphyxiation valve thereby communicating ambient air 17 into supply tube 40 (also see operational state 18 illustrated in FIG. 5C). In the FIG. 1 implementation, anti-asphyxiation valve is positioned between bag 43 and body 20. Anti-asphyxiation valve 70 may be omitted in other implementations, for example, because the user may breathe through the mouth in the event of an interruption in supply of respiratory gas 11 from gas source 99. An anti-asphyxiation valve, such as anti-asphyxiation valve 70, may be disposed about body 20 to communicate with body chamber 25 such as insert 60, in other implementations. Note that nasal cannula 26a, 26b occlude the nares with the mouth being unencumbered by nasal cannula apparatus 10, so that the user may always breath through the mouth if physically capable to do SO.

As illustrated in FIG. 1, bag 43 defines bag reservoir 45 sized to supply sufficient respiratory gas 11 during inhalation. Bag 43 may be formed of a compliant fluid-impermeable material such as polyethylene sheeting, and bag 43 may transition between a collapsed state which may occur in later portions of user inhalation when respiratory gas 11 is generally withdrawn from bag reservoir 45, and an expanded state, which may occur proximate completion of user exhalation when bag reservoir 45 is generally filled with respiratory gas 11.

As illustrated in FIG. 1, discharge tube 50 communicates outflow gas 13 from body chamber 25 of body 20 for discharge to the ambient environment. As illustrated, discharge tube 50 is terminated by connector 77 that allows for connection, inter alia, to package 80 (illustrated schematically in FIG. 1). Package 80 may variously include, for example, filter 82, sensor 84, PEEP valve 86. For example, in various implementations, connector 77 may be configured as a Luer-lock fitting.

Package 80, as illustrated in FIG. 1, cooperates with connector 77 to communicate outflow gas 13 from discharge tube 50 through package 80. As illustrated, package 80 includes filter 82 that cooperates with outflow gas 13 to remove pathogens from outflow gas 13.

Pathogens, as used herein, may include, for example, viruses, bacteria, and fungi, as well as, for example, bodily fluids and various noxious, odiferous, deleterious, or undesirable substances as may be included in outflow gas 13. Filter 82 may be omitted, in some implementations. Filter 82 may include various filter(s), activated carbon, and disinfectant(s), as would be readily recognized by those of ordinary skill in the art upon study of this disclosure. Package 80 may be omitted, in certain implementations. Discharge tube 50 may be omitted in implementations not including package 80, with outflow gas 13 thus being discharged through outflow port 23 into the ambient environment.

As illustrated in FIG. 1, package 80 includes sensor 84 in operable communication with outflow gas 13 to monitor attribute 44 (see FIG. 5B) of the outflow gas 13, as illustrated. Attribute 44 may include, for example, EtCO₂ (end tidal carbon dioxide indicative of adequacy of ventilation), FENO (exhaled nitric oxide indicative of airway inflammation, pulmonary hypertension and cardiac failure), other metabolic substances such as ketones in diabetic keto acidosis, and carbon monoxide. For example, attribute 44 may include changes in breathing cycle that, for example, may indicate hypopnea, and attribute 44 may indicate loss of pressure that may be indicative of apnea or a loose nasal cannula(s), such as nasal cannula 26a, 26b. Attribute 44, for example, may include temperature of outflow gas 13. Sensor 84 may be configured for digital operation and may communicate digitally via various wired or wireless technologies, in various implementations.

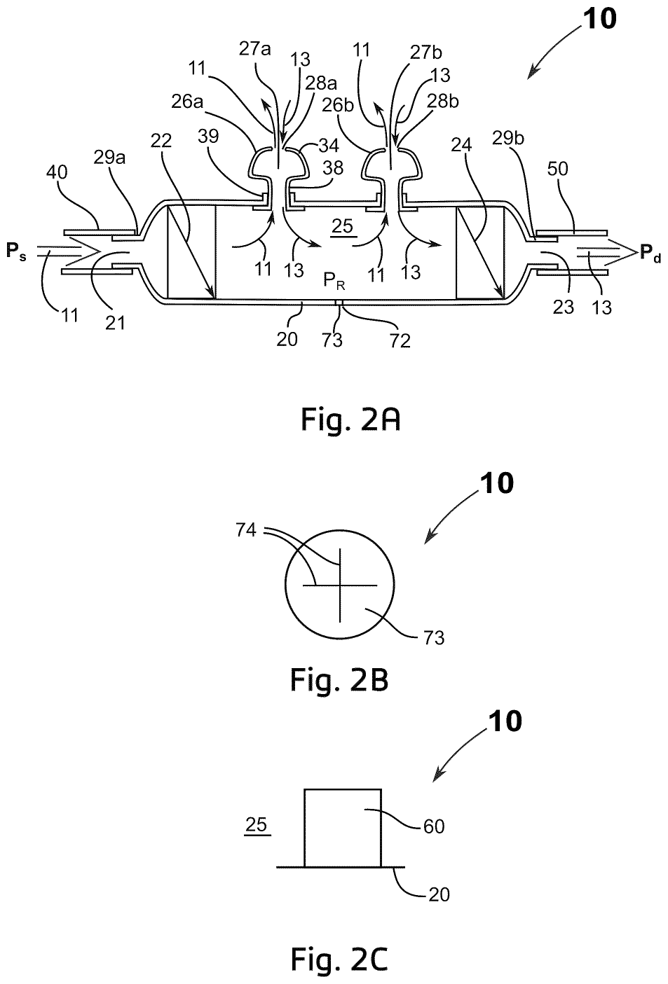

As illustrated in FIG. 1, package 80 includes PEEP valve 86 in fluid communication with outflow gas 13 to maintain a selected baseline pressure p_{BL} within body chamber 25 as the user exhales. That is, chamber pressure P_{R} (see FIG. 2A) within body chamber 25 is maintained at or above baseline pressure p_{BL}. Baseline pressure p_{BL} may be selected in order to maintain pressure on the most distal airways sufficient to prevents alveoli from collapsing during exhalation. Alveoli collapse may occur normally from absorption of oxygen in the alveolar sacs, and, unless these sacs are distended open, a ventilation perfusion mismatch and shunting develop resulting in loss of gas exchange ability. In ARDS (acute respiratory distress syndrome), loss of lung compliance may necessitate the use of PEEP valve 86 to improve oxygenation. PEEP valve 86 may be adjusted, for example, to select baseline pressure p_{BL} between about 5 cm to about 25 cm of water. Exemplary PEEP valves are manufactured, for example, by Becton Dickinson and Company of Franklin Lakes, NJ, Ambu A/S of Denmark, or Besmed of New Taipei City, Taiwan.

Thus, in exemplary nasal cannula apparatus 10, outflow gas 13 passes from body chamber 25 through discharge tube 50 and, thence, into package 80 that is connected to discharge tube 50 by mechanical cooperation with connector 77. Outflow gas 13 then passes through filter 82, then engages sensor 84, and, finally, outflow gas 13 passes through PEEP valve 86 and is discharged into the ambient environment from PEEP valve 86. Filter 82, sensor 84, and PEEP valve 86 may be arranged in other orders with respect to the flow of outflow gas 13, in various other implementations. Package 80, filter 82, sensor 84, and PEEP valve 86 are each optional, and, thus, any or all may or may not be included, in various implementations.

Supply tube 40, discharge tube 50, and supply pathway 47 of exemplary nasal cannula apparatus 10 may include various pipe(s), hose(s), fittings, connector(s), and other fluid conveyances, as would be readily understood by those of ordinary skill in the art upon study of this disclosure. For example, supply tube 40, discharge tube 50, and supply pathway 47 may, at least in part, be comprised of soft PVC tubing.

As illustrated in FIGS. 1 & 2C, insert 60 is insertably received by body 20 with portions disposed within body chamber 25 to cooperate with respiratory gas 11 and/or outflow gas 13.

Insert 60 may be permanently received by body 20 in some implementations, or insert 60 may be removably received to be replaceable in other implementations. Insert 60 may be variously configured to deliver aromatics or medicaments which may be of a sustained release type or thermally activated type. Insert 60 may be configured as a sensor that detects temperature, pressures, flow rate, quantity of exhaled gases such as CO₂, or quantities of certain exhaled metabolic substances such as ammonia, acetone, hydrogen cyanide, alcohols, pentane, acetic acid, methane, and sulphur compounds. Accurate quantification of these volatile trace metabolic substances in exhaled breath may aid clinical diagnostics. The rich vascular plexus in the nasal mucosa may enable photometric analysis of blood levels of oxygen, oxyhemoglobin and other parameters by insert 60. For example, heart rate, respiratory rate, or SpO₂, may be measured by insert 60 using nasal mucosal color. Heart rate and respiratory rate may be measured by insert 60 using MEMS-sensed rapid changes in pressure oscillations equal to heart beating against the airway vs slower and greater changes in pressure related to breathing. These two patterns are widely different so as to be readily differentiated.

As illustrated in FIG. 2A, nasal cannula 26a, 26b are affixed to body 20 with passages 27a, 27b in fluid communication with body chamber 25. Passages 27a, 27b communicate with the nares including the nasal passages through distal openings 28a, 28b, respectively, as illustrated. For example, nasal cannula 26a, 26b may be formed of an elastomer of Shore durometer between 5 to 50 on the 00 Shore hardness scale to maintain the user's comfort. Respiratory gas 11 may be communicated from body chamber 25 into the nares via passages 27a, 27b as the user inhales, and outflow gas 13 may be communicated from the nares into body chamber 25 via passages 27a, 27b as the user exhales.

Supply tube 40 is affixed to connector 29a that defines inflow port 21 through which respiratory gas 11 flows from supply tube 40 into body chamber 25, and discharge tube 50 is affixed to connector 29b that defines outflow port 23 through which outflow gas 13 flows from body chamber 25 into discharge tube 50, as illustrated in FIG. 2A. Connectors 29a, 29b are illustrated as nipples but may assume various other configurations, in various other implementations. In certain implementations, supply tube 40, body 20, and discharge tube 50 may be of unitary construction thus making connectors 29a, 29b superfluous. Inflow valve 22, illustrated schematically in FIG. 2A, operates as a check valve that regulates the inflow of respiratory gas 11 from supply tube 40 through inflow port 21 into body chamber 25. Outflow valve 24, also illustrated schematically in FIG. 2A, operates as a check valve that regulates outflow from body chamber 25 through outflow port 23 into discharge tube 50. In implementations omitting discharge tube 50, outflow valve 24 regulates outflow from body chamber 25 through outflow port 23 into the ambient environment.

Arrows in FIG. 2A illustrate exemplary flows of respiratory gas 11 including from supply tube 40 at supply pressure pₛ into body chamber 25 through inflow port 21 regulated by inflow valve 22 and from body chamber 25 into the nares via passages 27a, 27b of nasal cannula 26a, 26b, respectively. Arrows in FIG. 2A also illustrate exemplary flows of outflow gas 13 from the nares into body chamber 25 via passages 27a, 27b of nasal cannula 26a, 26b, respectively, and thence from body chamber 25 through outflow port 23 regulated by outflow valve 24 into discharge tube 50 with discharge pressure p_{d}. As illustrated in FIG. 2A, chamber pressure P_{R} refers to pressure within body chamber 25, supply pressure pₛ refers to pressure within supply tube 40 proximate inflow port 21, and discharge pressure p_{d} refers to pressure within discharge tube 50 proximate outflow port 23. Discharge pressure p_{d} may be generally equal to ambient pressure p_{amb}, in certain implementations.

As illustrated in FIG. 2A, body 20 includes drain port 72 passing though body 20 at a bottom portion thereof. Drain port 72 is configured to drain liquids that may accumulate within body chamber such as condensation or various bodily fluids. Membrane 73 may be disposed within drain port 72 to control the passage of liquids therethrough. For example, slits 74 (illustrated in FIG. 2B) in membrane 73 may flex when chamber pressure P_{R} exceeds ambient pressure p_{amb} during exhalation thereby allowing liquids to flow out of body chamber 25 through drain port 72.

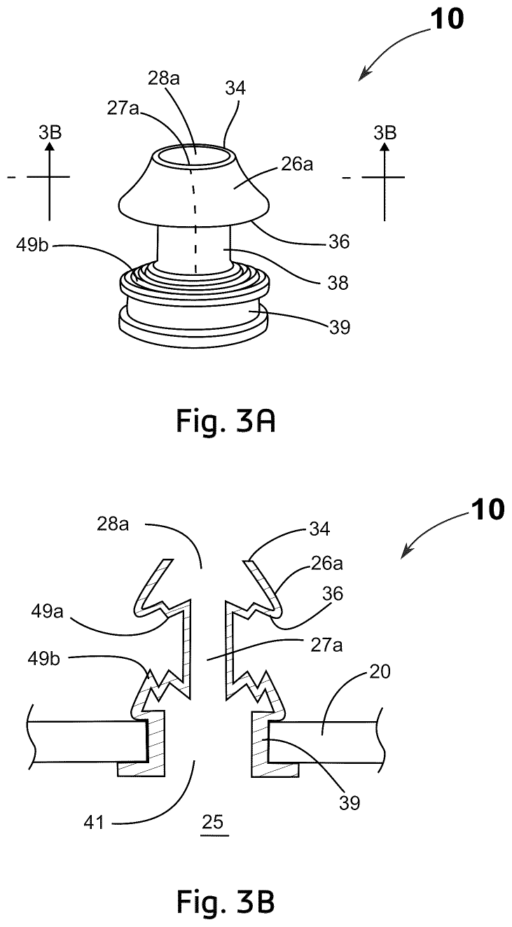

FIGS. 3A, 3B illustrate an exemplary embodiment of a nasal cannula 26a, with nasal cannula 26b being formed in a similar manner in exemplary nasal cannula apparatus 10. As illustrated in FIGS. 3A, 3B, nasal cannula 26a, is tapered proximate distal end 34 and broader proximate proximal end 36 thus being adapted to comfortably and ergonomically fit against a naris. Note that the surface of nasal cannula 26a may have a straight or a slightly concave or slightly convex profile between distal end 34 and proximal end 36, in various implementations. When viewed from the top, nasal cannula 26a may additionally have an ovoid or circular circumference with circular being illustrated in FIGS. 3A, 3B. Nasal cannula 26a has a distal opening 28a which communicates fluidly via passage 27a with body chamber 25 inside body 20. Nasal cannula 26a sits atop a soft elastomer stem 38 that joins retention structure 39 adapted to be secured through aperture 41 formed in body 20 (FIG. 3B) thus securely engaging mechanically nasal cannula 26a with body 20. Optional features of nasal cannula 26a include concentric, accordion-like corrugations 49a underneath proximal end 36 or similar corrugations 49b proximate the joining of stem 38 with retention structure 39, as illustrated. Corrugations 49a, 49b may incrementally add compressional and tilting flexibility to nasal cannula 26a thereby according a more occlusal fit with the naris and reduce the risk of focal soft-tissue compression injury against the naris. Making nasal cannula 26a using an elastomer of sufficient softness (such as soft or extra-soft Shore durometer, for example, in a range generally between 5-50 on the 00 Shore hardness scale) may obviate the need for the optional corrugation features.

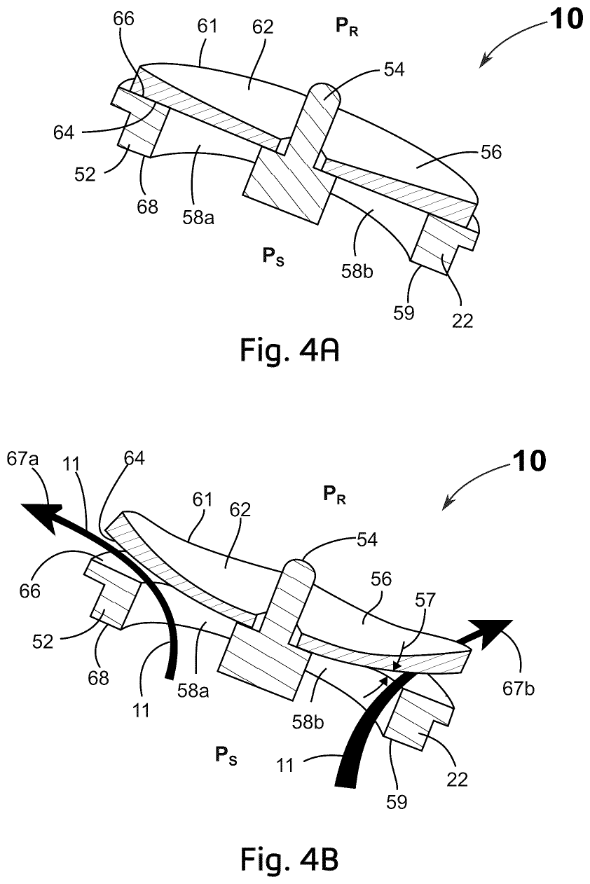

An exemplary implementation of inflow valve 22 is illustrated in FIGS. 4A, 4B. As illustrated, inflow valve 22 is configured as a check valve and includes valve member 56 received over pin 54 that extends forth from valve seat 52 to detain valve member 56 in cooperation with valve seat 52. Outflow valve 24 may be formed and operate similarly to exemplary inflow valve 22. Of course, inflow valve 22 and outflow valve 24 may variously differ from one another or may be otherwise configured, in various other implementations, as would be readily recognized by those of ordinary skill in the art upon study of this disclosure. An outer perimeter of valve seat 52, for example, may be secured within body chamber 25 in cooperation with inflow port 21 to control the inflow of respiratory gas 11 into chamber 25. Valve apertures, such as valve apertures 58a, 58b, formed in exemplary valve seat 52 allow respiratory gas 11 flow through valve seat 52 via the valve apertures. As illustrated, inflow valve 22 is oriented so that surface 62 of valve member 56 is on the downstream side 61 of inflow valve 22 and surface 68 of valve seat 52 is on the upstream side 59 of inflow valve 22. That is, pin 54 is oriented to extend forth from valve seat 52 in a flow direction of respiratory gas 11 e.g., away from supply tube 40 toward body chamber 25 for inflow valve 22, in this implementation. Outflow valve 24 has pin, such as pin 54, oriented away from body chamber 25 toward outflow port 23 and discharge tube 50 so that flow passes through outflow valve 24 from body chamber 25 through outflow port 23 into discharge tube 50.

Inflow valve 22 is positionable between CLOSED position illustrated in FIG. 4A and OPEN position illustrated in FIG. 4B. In CLOSED position illustrated in FIG. 4A, chamber pressure p_{R} within body chamber 25 on downstream side 61 of inflow valve 22 is greater than supply pressure pₛ within supply tube 40 on upstream side 59 of inflow valve 22 thereby holding portions of surface 64 of valve member 56 in biased engagement with portions of surface 66 of valve seat 52. The biased sealing engagement of portions of surface 64 with portions of surface 66 sealingly engages valve member 56 with valve seat 52 thus blocking flow of respiratory gas 11 through inflow valve 22 into body chamber 25, in this implementation. Chamber pressure P_{R} within body chamber 25 is greater than supply pressure pₛ within supply tube 40, for example, during user exhalation. Thus, user exhalation may position inflow valve 22 in CLOSED position blocking inflow of respiratory gas 11 into body chamber 25 during exhalation.

In OPEN position illustrated in FIG. 4B, supply pressure pₛ within supply tube 40 is greater than chamber pressure p_{R} within body chamber 25 thereby causing portions of surface 64 of valve member 56 to flex into spaced relation with portions of surface 66 of valve seat 52. Because portions of surface 64 are in spaced relation with portions of surface 66 in OPEN position, respiratory gas 11 flows through inflow valve 22 from upstream side 59 (e.g., supply tube 40) to downstream side 61 (e.g., body chamber 25) by flowing through valve apertures, such as valve apertures 58a, 58b, formed in valve seat 52 and through gap 57 between portions of surface 64 of valve member 56 and portions of surface 66 of valve seat 52, as indicated by arrows 67a, 67b in FIG. 4B. Body chamber pressure p_{R} within body chamber 25 is less than supply pressure pₛ within supply tube 40, for example, during user inhalation. Thus, user inhalation positions inflow valve 22 in the OPEN position allowing inflow of respiratory gas 11 into body chamber 25 during inhalation.

Valve seat 52 may be made of hard plastic, and valve member 56 may be made of a soft, flexible material. For example, valve member 56 may be variously formed of rubber, polysiloxane, or other silicone polymers including dimethicone, cyclopentasiloxane, dimethiconol, phenyl trimethicone, amodimethicone, and cyclomethicone. Valve member 56 may have a durometer value (e.g., Shore hardness) within a selected range of durometer values that enable proper operation of the valve. For example, valve member 56 may have a Shore hardness within a range of from about 40 to about 60 on the 00 scale.

Outflow valve 24 is in CLOSED position when chamber pressure P_{R} within body chamber 25 is less than discharge pressure p_{d} within discharge tube 50, for example, during user inhalation. Thus, flow from body chamber 25 into discharge tube 50 is blocked by outflow valve 24 during inhalation. Outflow valve 24 in in the OPEN position when chamber pressure P_{R} within body chamber 25 is greater than discharge pressure p_{d} within discharge tube 50, for example, during user exhalation. Thus, outflow gas 13 flows through outflow valve 24 from body chamber 25 into discharge tube 50 during exhalation, in this implementation.

In operation, exemplary nasal cannula apparatus 10 may variously transition between exemplary operational states 14, 16, 18 illustrated in FIGS. 5A, 5B, 5C, respectively, depending upon the supply pressure pₛ within supply tube 40, the chamber pressure P_{R} within body chamber 25, and discharge pressure p_{d} within discharge tube 50 as prompted by user inhalation and exhalation. For example, during user inhalation, supply pressure pₛ within supply tube 40 is greater than chamber pressure P_{R} within body chamber 25 due to inhalation from body chamber 25 thereby placing inflow valve 22 in OPEN position allowing respiratory gas 11 to flow from supply tube 40 into body chamber 25. During user inhalation of respiratory gas 11, chamber pressure P_{R} is less than discharge pressure p_{d} within discharge tube 50 placing outflow valve 24 in CLOSED position blocking flow between body chamber 25 and discharge tube 50 so that there is neither flow of respiratory gas 11 from body chamber 25 into discharge tube 50 nor back flow of outflow gas 13 from discharge tube 50 into body chamber 25. During user exhalation of outflow gas 13, for example, chamber pressure pr in body chamber 25 is greater than supply pressure pₛ within supply tube 40 placing inflow valve 22 in CLOSED position blocking the respiratory gas 11 from flowing into body chamber 25 from supply tube 40 to dilute outflow gas 13 within body chamber 25 or waste respiratory gas 11. Chamber pressure P_{R} is greater than discharge pressure p_{d} within discharge tube 50 during user exhalation thereby placing outflow valve 24 in OPEN position allowing flow of outflow gas 13 from body chamber 25 into discharge tube 50.

In operational states 14, 16, 18 of exemplary nasal cannula apparatus 10, body 20 is positioned generally between the upper lip and the external nares of the user with nasal cannula 26a, 26b operably engaged with the nares of the user in operational states 14, 16, 18. Straps 33a, 33b may maintain body 20 and nasal cannula 26a, 26b in position. It is assumed, in this example, that source 99 continuously provides a constant flow of respiratory gas 11 in operational states 14, 16.

In exemplary operational state 14 of exemplary nasal cannula apparatus 10 illustrated in FIG. 5A, the user is inhaling respiratory gas 11 through nasal cannula 26a, 26b that is being delivered from source 99. As the user inhales respiratory gas 11 from body chamber 25 through nasal cannula 26a, 26b the chamber pressure P_{R} within body chamber 25 decreases to less than supply pressure pₛ within supply tube 40 thereby positioning inflow valve 22 in the OPEN position. As illustrated in FIG. 5A, respiratory gas 11 is communicated from source 99 into bag reservoir 45 of bag 43 via supply pathway 47 in operational state 14. Respiratory gas 11 is then communicated from bag reservoir 45 via supply tube 40 into body chamber 25 through inflow port 21 as inflow valve 22 is in the OPEN position, and, thence, into the nares through nasal cannula 26a, 26b in operational state 14. Bag reservoir 45 provides additional respiratory gas 11 to that being provided by source 99 in order to meet PIFR, so that the quantity of respiratory gas 11 within bag reservoir 45 may be being depleted during operational state 14.

Outflow valve 24 is in a CLOSED position preventing flow through outflow port 23 from body chamber 25 in operational state 14. Outflow valve 24 is positioned in the CLOSED position by the chamber pressure P_{R} within body chamber 25 being less than discharge pressure p_{d} within the discharge tube 50 due to the user inhaling respiratory gas 11 from body chamber 25 in operational state 14. Thus, there is no loss of respiratory gas 11 by flow of respiratory gas 11 into discharge tube 50 in operational state 14. Moreover, outflow gas 13 in discharge tube 50 is not diluted by respiratory gas 11 wherein, for example, such dilution may compromise accurate detection of attribute 44 in outflow gas 13 by sensor 84. Note that anti-asphyxiation valve 70 that cooperates fluidly with supply tube 40 is in the CLOSED position in operational state 14.

In exemplary operational state 16 of exemplary nasal cannula apparatus 10 illustrated in FIG. 5B, the user is exhaling outflow gas 13 through nasal cannula 26a, 26b into body chamber 25. Outflow valve 24 is in an OPEN position allowing communication of outflow gas 13 from body chamber 25 through outflow port 23 into discharge tube 50 by the chamber pressure P_{R} within body chamber 25 being greater than discharge pressure p_{d} within the discharge tube 50. Discharge tube 50 communicates the outflow gas 13 to package 80 where outflow gas 13 is filtered by filter 82 and sampled by sensor 84 before being discharged into the ambient environment, in this implementation. PEEP valve 86 is also included in package 80, in this implementation.

Inflow valve 22 is in a CLOSED position blocking flow of respiratory gas 11 into body chamber 25 through inflow port 21 in operational state 16, as illustrated in FIG. 5B. There is no flow of respiratory gas 11 from supply tube 40 into discharge tube 50 via body chamber 25 that dilutes outflow gas 13, in operational state 16. Thus, sensor 84 detects attribute 44 in outflow gas 13 undiluted by respiratory gas 11, which may increase the accuracy with which attribute 44 is detected. In operational state 16, respiratory gas 11 from source 99 flows into bag reservoir 45 to refill bag reservoir 45 that was depleted during operational state 14. In certain implementations, a relief valve (not shown) may be provided that cooperates fluidly with bag reservoir 45 to prevent overfilling of bag reservoir 45. Note that anti-asphyxiation valve 70 is in the CLOSED position in operational state 16. Sensor 84 detects attribute 44 in outflow gas 13 at least during operational state 16.

Exemplary nasal cannula apparatus 10 alternates between operational states 14, 16 as the user inhales and exhales, respectively. Accordingly the user exhales into body chamber 25 and then inhales from body chamber 25 that may result in the user inhaling outflow gas 13 from body chamber 25 that the user previously exhaled. In various implementations, the body chamber, such as body chamber 25, 125, may have a volume generally within a range of about 2 ml to about 10 ml. For example, for a body chamber having a volume of 10 ml, the user may then inhale 10 ml of previously exhaled outflow gas from the body chamber that would be included in a tidal volume of 450 ml resulting in about 2% of the tidal volume being inhaled including outflow gas. Per this example, about 98% of the outflow gas is discharged from the body chamber without being re-inhaled. A 2 ml volume of the body chamber is about 0.4% of a 450 ml tidal volume.

In exemplary operational state 18 of exemplary nasal cannula apparatus 10 illustrated in FIG. 5C, the user is inhaling through nasal cannula 26a, 26b but source 99 has failed and bag reservoir 45 is depleted so that there is insufficient respiratory gas 11 available. User inhalation when available respiratory gas 11 is insufficient positions inflow valve 22 in OPEN position and decreases both supply pressure pₛ in supply tube 40 and chamber pressure P_{R} within body chamber 25 to less than trigger pressure pₓ below ambient pressure p_{amb} (e.g., pₛ≈p_{R}≤pₓ<p_{amb}) thereby positioning anti-asphyxiation valve 70 into the OPEN position from the CLOSED position. That is, anti-asphyxiation valve 70 opens at a pressure at least pₓ below ambient pressure p_{amb}. Anti-asphyxiation valve 70 in OPEN position allows communication of ambient air 17 into supply tube 40, as illustrated in FIG. 5C. Ambient air 17 is then communicated from anti-asphyxiation valve 70 by supply tube 40 into body chamber 25 through inflow port 21 as inflow valve 22 is in the OPEN position, and, thence, into the nares through nasal cannula 26a, 26b in operational state 18, as illustrated. Outflow valve 24 is in the CLOSED position preventing flow out of outflow port 23 from body chamber 25 in exemplary operational state 18. Operational state 18 provides a safety measure for users unable to shift from nasal breathing to mouth breathing should sufficient respiratory gas 11 become unavailable.

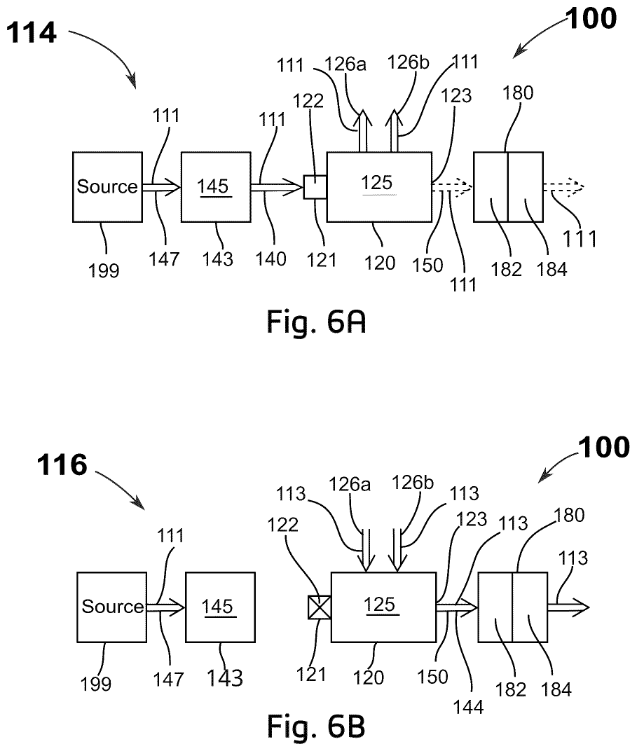

Another exemplary nasal cannula apparatus 100 is illustrated in FIGS. 6A, 6B, respectively. Body 120 of exemplary nasal cannula apparatus 100 defines body chamber 125 generally similarly to body chamber 25 of body 20 of exemplary nasal cannula apparatus 10. Supply pathway 147, supply tube 140, and discharge tube 150 of exemplary nasal cannula apparatus 100 are generally equivalent to supply pathway 47, supply tube 40, and discharge tube 50, respectively, of exemplary nasal cannula apparatus 10. Bag 143 with bag reservoir 145 of nasal cannula apparatus 100 is generally equivalent to bag 43 with bag reservoir 45 of nasal cannula apparatus 10. Bag 143 is optional and may be omitted from certain implementations. As illustrated, nasal cannula apparatus 100 includes package 180 that includes filter 182 and sensor 184 all generally equivalent to package 80, filter 82, and sensor 84 of nasal cannula apparatus 10. It is assumed in this exemplary implementation that source 199 of nasal cannula apparatus 100 continuously provides a constant flow of respiratory gas 111. A PEEP valve, such as PEEP valve 86, and an anti-asphyxiation valve, such as anti-asphyxiation valve 70, are both omitted in exemplary nasal cannula apparatus 100 as these are optional as are package 180, filter 182, and sensor 184.

Nasal cannula 126a, 126b communicate fluidly between the nares and body chamber 125 and may be formed similarly to nasal cannula 26a, 26b of exemplary nasal cannula apparatus 10. Body 120 includes inflow port 121 through which respiratory gas 111 may be communicated from source 199 into body chamber 125 via supply tube 140. Body 120 includes outflow port 123 through which outflow gas 113 is communicated out of body chamber 125 into discharge tube 150. In this implementation, outflow gas 113 is communicated through discharge tube 150, through filter 182 and sensor 184 of package 180, and then discharged into the ambient environment. In implementations omitting package 180, discharge tube 150 may also be omitted and outflow gas communicated out of body chamber 125 through outflow port 123 into the ambient environment. Note that, in exemplary nasal cannula apparatus 100, inflow port 121 is controlled by inflow valve 122 and outflow port 123 is uncontrolled by a valve in contrast with exemplary nasal cannula apparatus 10 wherein outflow port 23 is controlled by outflow valve 24. That is, only one valve, inflow valve 122, controls fluid communication with body chamber 125 in exemplary nasal cannula apparatus 100. There is no valve controlling fluid communication between body chamber 125 and discharge tube 150, in this implementation.

In operation of exemplary nasal cannula apparatus 100, body 120 is positioned generally between the upper lip and the external nares of the user with nasal cannula 126a, 126b operably engaged with the nares of the user in operational states 114, 116. Body 120 may contact the skin of the user generally between the upper lip and the external nares. With nasal cannula apparatus 100 so engaged with the user, nasal cannula apparatus 100 may transition between exemplary operational states 114, 116 illustrated in FIGS. 6A, 6B, respectively, as the user inhales and exhales. In exemplary operational state 114 illustrated in FIG. 6A, the user is inhaling respiratory gas 111 delivered from source 199 and from bag reservoir 145 through nasal cannula 126a, 126b. As illustrated in FIG. 6A, respiratory gas 111 is communicated from source 199 into bag reservoir 145 of bag 143 via supply pathway 147. Respiratory gas 111 is then communicated from bag reservoir 145 via supply tube 140 into body chamber 125 through inflow port 121 as inflow valve 122 is in an OPEN position, and, thence, into the nares through nasal cannula 126a, 126b from body chamber 125. Because outflow port 123 is unregulated, there may be some flow of respiratory gas 111 from body chamber 125 into discharge tube 150 through outflow port 123 in operational state 114 (indicated by the phantom arrow in FIG. 6A) that may then be communicated to sensor 184. This loss of respiratory gas 111 into discharge tube 150 may be minimal as most if not all respiratory gas 111 may be being inhaled by the user particularly as bag reservoir 145 is being depleted. Respiratory gas 111 in discharge tube 150, if any, may then pass through package 180 where it is sampled by sensor 184 before being expelled to the ambient environment.

In exemplary operational state 116 illustrated in FIG. 6B, the user is exhaling outflow gas 113 through nasal cannula 126a, 126b into body chamber 125. Outflow gas 113 is then communicated from body chamber 125 through outflow port 123 into discharge tube 150. Discharge tube 150 communicates outflow gas 113 to package 180 where outflow gas 113 is filtered by filter 182 and sampled by sensor 184 before being discharged into the ambient environment from package 180, in this implementation. Sensor 184 detects attribute 144 in outflow gas 113 at least during operational state 116.

In operational state 116, inflow valve 122 is in a CLOSED position blocking flow of respiratory gas 111 into body chamber 125 through inflow port 121 during exhalation, as illustrated in FIG. 6B. Thus, in operational state 116, there is no flow of respiratory gas 111 from supply tube 140 into discharge tube 150 that dilutes outflow gas 113 during exhalation. In operational state 116, sensor 184 detects attribute 144 in outflow gas 113 undiluted by respiratory gas 111 at least during user exhalation, so that attribute 144 may be accurately detected by sensor 184 at least during exhalation. Respiratory gas 111 from source 199 flows into bag reservoir 145 of bag 143 in operational state 116 to refill bag reservoir 145 that was depleted during operational state 114.

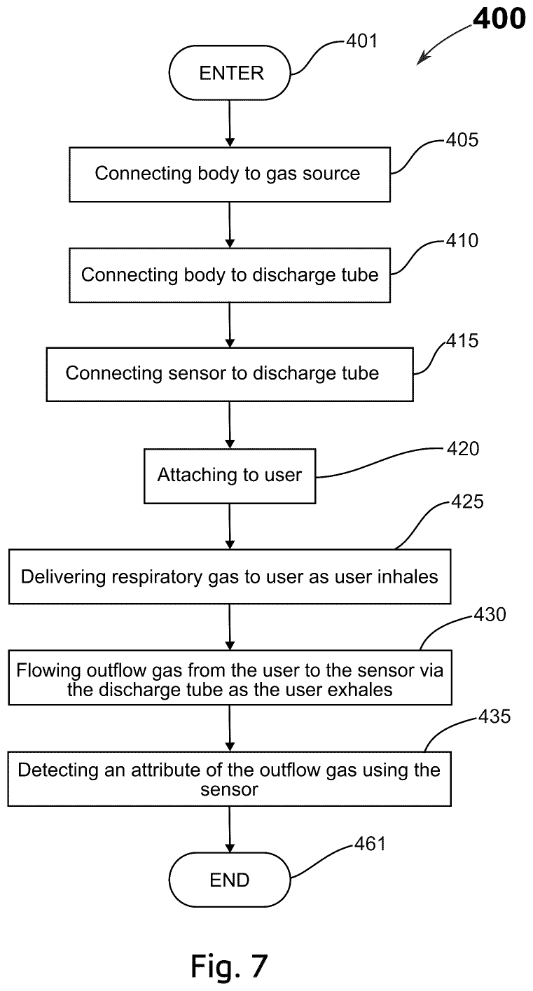

Exemplary method 400 of operating a respiratory support apparatus, such as exemplary respiratory support apparatus 10, 100, is illustrated in FIG. 7. Method 400 is exemplary, so that the steps of method 400 may be combined, further discretized, performed in various other orders, omitted, or additional steps may be included, in other implementations.

Method 400 is entered at step 401. At step 405, a supply tube, such as supply tube 40, 140, is placed in cooperation with the body, such as body 20, 120, to fluidly communicate respiratory gas, such as respiratory gas 11, 111, from a gas source, such as gas source 99, 199 with a body chamber, such as body chamber 25, 125, of the body. A bag having a bag reservoir, such as bag 43, 143 with corresponding bag reservoir 45, 145, may be interposed between the gas source and the body chamber to communicate respiratory gas received from the gas source with the supply tube. The bag reservoir, when present, may assist in meeting PIFR.

At step 410, a discharge tube, such as discharge tube 50, 150, is placed in communication with the body chamber to communicate outflow gas, such as outflow gas 13, 113, from the body chamber.

At step 415, a package, such as package 80, 180, that includes a sensor, such as sensor 84, 184, is placed in communication with the discharge tube. The sensor is configured to detect an attribute, such as attribute 44, 144, of the outflow gas.

At step 420, the body is attached to the user. The body may be placed generally between the upper lip and the external nares of the user with nasal cannulae, such as nasal cannula 26a, 26b, 126a, 126b, operably engaged with the nares of the user. The nasal cannulae are in communication with the body chamber.

At step 425 as the user inhales respiratory gas through the nasal cannulae for delivery to the lungs, the chamber pressure P_{R} within the body chamber decreases to less than supply pressure pₛ within the supply tube thereby positioning an inflow valve, such as inflow valve 22, 122, in the OPEN position flowing the respiratory gas from the source via the supply tube, through the body chamber, and then through the nasal cannulae into the nares. As the user inhales, the chamber pressure pr within the body chamber decreases to less than discharge pressure p_{d} within the discharge tube thereby positioning an outflow valve, such as outflow valve 24, if present, in the CLOSED position blocking fluid communication between the body chamber and the discharge tube. Blocking fluid communication between the body chamber and the discharge tube during inhalation may prevent inhalation of the outflow gas from the discharge tube thereby diluting the respiratory gas being delivered to the user via the nares. Blocking fluid communication between the body chamber and the discharge tube during inhalation may prevent dilution of the outflow gas in the discharge tube with respiratory gas thereby interfering with accurate measurement of the attribute of the outflow gas by the sensor. Note that respiratory gas communicated into the discharge tube during inhalation is generally wasted.

At step 430, as the user exhales outflow gas from the lungs through the nasal cannulae into the body chamber, the chamber pressure P_{R} within the body chamber increases to greater than supply pressure pₛ within the supply tube thereby positioning the inflow valve in the CLOSED position blocking the flow respiratory gas from the supply tube into the body chamber. Respiratory gas communicated into the body chamber during exhalation is generally wasted. As the user exhales, the chamber pressure P_{R} within the body chamber increases to greater than discharge pressure p_{d} within the discharge tube thereby positioning the outflow valve, if present, in the OPEN position allowing the flow of outflow gas from the body chamber into the discharge tube. If the outflow valve is not present, the outflow gas flows from the body chamber into the discharge tube.

At step 435, the sensor detects the attribute of the outflow gas as the outflow gas is flowing through the discharge tube during user exhalation. The sensor may communicate data indicative of the attribute to a computer for processing and display, and may do so generally in real time either intermittently or continuously. The computer may be used to control the sensor.

Method 400 terminates at step 461. The respiratory support apparatus is assembled by steps 405, 410, 415 in this exemplary implementation. The respiratory support apparatus may be provided in various states of assembly so that steps 405 may be omitted or additional steps may be required, in other implementations. Steps 425, 430, 435 repeat cyclically, in various implementations. Step 435 may occur generally simultaneous with step 430, in various implementations.

The foregoing discussion along with the Figures discloses and describes various exemplary implementations. These implementations are not meant to limit the scope of coverage, but, instead, to assist in understanding the context of the language used in this specification and in the claims. The Abstract is presented to meet requirements of 37 C.F.R. § 1.72(b) only. Accordingly, the Abstract is not intended to identify key elements of the apparatus and methods disclosed herein or to delineate the scope thereof. Upon study of this disclosure and the exemplary implementations herein, one of ordinary skill in the art may readily recognize that various changes, modifications and variations can be made thereto without departing from the spirit and scope of the inventions as defined in the following claims.

The invention claimed is:

1. A nasal cannula apparatus, comprising:
a body that defines a body chamber, the body adapted to engage a user between nares and upper lip;
a pair of nasal cannula in cooperation with the body, each nasal cannula of the pair of nasal cannula adapted to engage occlusively a naris in order to communicate fluidly between the naris and the body chamber via the nasal cannula;
an inflow port in fluid communication with the body chamber;
an inflow valve disposed within the body chamber in cooperation with the inflow port to control communication of a respiratory gas through the inflow port into the body chamber to be inhaled via the pair of nasal cannula;
an outflow port in fluid communication with the body chamber to communicate an outflow gas from the body chamber, the outflow gas having been exhaled into the body chamber via the pair of nasal cannula; and
wherein the inflow valve allows respiratory gas communication through the inflow port into the body chamber during inhalation and blocks respiratory gas communication through the inflow port into the body chamber during exhalation.

2. The apparatus of claim 1, further comprising:
an outflow valve disposed within the body chamber in cooperation with the outflow port to control fluid communication through the outflow port.

3. The apparatus of claim 2, wherein the outflow valve blocks fluid communication through the outflow port during inhalation and allows fluid communication through the outflow port during exhalation.

4. The apparatus of claim 1, further comprising:
a sensor in fluid cooperation with the outflow port to detect an attribute of the outflow gas communicated through the outflow port, the outflow gas having been exhaled.

5. The apparatus of claim 4, the sensor configured to detect the attribute only during exhalation.

6. The apparatus of claim 4, the attribute is selected from a group consisting of carbon dioxide, nitric oxide, a ketone, carbon monoxide, and a temperature.

7. The apparatus of claim 1, further comprising:
a filter in fluid communication with the outflow port to filter the outflow fluid.

8. The apparatus of claim 1, further comprising:
a PEEP valve in fluid communication with the outflow port to maintain a chamber pressure within the body chamber above a baseline pressure.

9. The apparatus of claim 1, further comprising:
an anti-asphyxiation valve in communication with the body chamber to communicate ambient air into the body chamber when a chamber pressure within the body chamber falls below a trigger pressure.

10. The apparatus of claim 1, further comprising:
a bag defining a bag reservoir in fluid communication with the inflow port to communicate the respiratory gas from the bag chamber into the body chamber during inhalation.

11. A nasal cannula apparatus, comprising:
a body that defines a body chamber, the body adapted to engage a user generally above an upper lip;
a pair of nasal cannula attached to the body, each nasal cannula of the pair of nasal cannula adapted to engage occlusively a naris in order to communicate fluidly between the naris and the body chamber via the nasal cannula;
a supply tube in fluid communication with the body chamber to communicate a respiratory gas from a gas source into the body chamber for inhalation via the pair of nasal cannula;
an inflow valve in fluid cooperation with the supply tube and the body chamber to control communication of the respiratory gas from the supply tube into the body chamber;
a discharge tube in fluid communication with the body chamber to communicate an outflow gas from the body chamber, the outflow gas having been exhaled into the body chamber via the pair of nasal cannula; and
wherein the inflow valve allows communication of the respiratory gas from the supply tube into the body chamber during inhalation and blocks communication of the respiratory gas from the supply tube into the body chamber during exhalation.

12. The apparatus of claim 11, further comprising:
an outflow valve in fluid cooperation with the discharge tube and the body chamber to control fluid communication between the discharge tube and the body chamber.

13. The apparatus of claim 12, wherein the outflow valve blocks fluid communication between the discharge tube and the body chamber during inhalation and allows fluid communication from the body chamber into the discharge tube during exhalation.

14. The apparatus of claim 11, further comprising:
a sensor in fluid cooperation with the discharge tube to detect an attribute of the outflow gas.

15. The apparatus of claim 14, the sensor configured to detect the attribute only during exhalation.

16. A method of respiratory therapy, comprising the steps of:
attaching a body to the user generally between an upper lip and external nares with a pair of nasal cannula occlusively engaged with the nares of the user, the pair of nasal cannula being in fluid communication with a body chamber defined by the body;
decreasing a chamber pressure within the body chamber to less than a supply pressure within a supply tube communicating with the body chamber by inhaling respiratory gas from the body chamber through the pair of nasal cannula thereby positioning an inflow valve in an OPEN position flowing respiratory gas from the supply tube into the body chamber and thence via the pair of nasal cannula into the nares; and
increasing the chamber pressure within the body chamber to greater than the supply pressure within the supply tube by exhaling an outflow gas through the pair of nasal cannula into the body chamber thereby positioning the inflow valve in a CLOSED position blocking respiratory gas flow from the supply tube into the body chamber.

17. The method of claim 16, further comprising the step of:
positioning an outflow valve in a CLOSED position by decreasing the chamber pressure within the body chamber to less than a discharge pressure within a discharge tube communicating with the body chamber by inhaling respiratory gas from the body chamber through the pair of nasal cannula, the outflow valve in the CLOSED position blocking fluid communication between the body chamber and the discharge tube.

18. The method of claim 16, further comprising the step of:
positioning an outflow valve in an OPEN position by increasing the chamber pressure within the body chamber to greater than a discharge pressure within a discharge tube communicating with the body chamber by exhaling outflow gas through the pair of nasal cannula into the body chamber, the outflow valve in the OPEN position communicating the outflow gas from the body chamber into the discharge tube.

19. The method of claim 16, further comprising the step of:
detecting an attribute of the outflow gas using a sensor in fluid cooperation with the outflow gas.

20. The method of claim 19, wherein the attribute is selected from a group consisting of carbon dioxide, nitric oxide, a ketone, carbon monoxide, and a temperature.
